(19) United States
(12) Patent Application Publication
(10) Pub. No.: US 2025/0375103 A1
(43) Pub. Date: Dec. 11, 2025

(54) ENDOSCOPE SYSTEM

(71) Applicant: GUANGZHOU LUXVISIONS INNOVATION TECHNOLOGY LIMITED, Guangzhou City (CN)

(72) Inventor: Chih-Ju Lin, Guangzhou City (CN)

(73) Assignee: GUANGZHOU LUXVISIONS INNOVATION TECHNOLOGY LIMITED, Guangzhou City (CN)

(21) Appl. No.: 19/014,226

(22) Filed: Jan. 9, 2025

Related U.S. Application Data
(60) Provisional application No. 63/658,451, filed on Jun. 11, 2024.

(30) Foreign Application Priority Data
Sep. 23, 2024 (CN) 202411326631.2

(51) Int. Cl. A61B 1/07; A61B 1/00; A61B 1/06

(52) U.S. Cl. CPC A61B 1/07; A61B 1/00126; A61B 1/00197; A61B 1/0638

(57) ABSTRACT

An endoscope system including first and second light sources, first, second and third optical fibers, a first optical fiber coupler, a bidirectional coupler, and an optical fiber endoscope is provided. A wavelength of light emitted by the first light source is between 400 nm and 800 nm. The first optical fiber is connected to the first light source. A wavelength of light emitted by the second light source is between 900 nm and 1700 nm. The second optical fiber is connected to the second light source. The first optical fiber coupler is connected to the first and second optical fibers. The third optical fiber is connected to the first optical fiber coupler. The bidirectional coupler is connected to the third optical fiber. The optical fiber endoscope has a first end provided with a microlens array group, and a second end connected to the bidirectional coupler.

### CROSS-REFERENCE TO RELATED APPLICATION

This application claims the priority benefit of U.S. provisional application Ser. No. 63/658,451, filed on Jun. 11, 2024 and China application serial no. 202411326631.2, filed on Sep. 23, 2024. The entirety of each of the above-mentioned patent applications is hereby incorporated by reference herein and made a part of this specification.

### BACKGROUND

### Technical Field

The disclosure relates to an endoscope system, and particularly relates to an endoscope system adapted to provide multiple images within excellent quality.

### Description of Related Art

Current clinical endoscopes mainly adopt visible light images, and endoscopes having different diameters are used according to different parts and expected treatment methods. How to improve quality of endoscopic images of a diseased part to meet the needs of medical clinical images is a research direction of this field.

### SUMMARY

The disclosure is directed to an endoscope system, which uses a plurality of light sources with different wavelengths to improve quality of endoscopic images of a diseased part.

According to an embodiment of the disclosure, an endoscope system includes a first light source, a first optical fiber, a second light source, a second optical fiber, a first optical fiber coupler, a third optical fiber, a bidirectional coupler, and an optical fiber endoscope. A wavelength of light emitted by the first light source is between 400 nm and 800 nm. The first optical fiber is connected to the first light source. A wavelength of light emitted by the second light source is between 900 nm and 1700 nm. The second optical fiber is connected to the second light source. The first optical fiber coupler is connected to the first optical fiber and the second optical fiber. The third optical fiber is connected to the first optical fiber coupler. The bidirectional coupler is connected to the third optical fiber. The optical fiber endoscope has a first end and a second end, the first end is provided with a microlens array group, and the second end is connected to the bidirectional coupler.

In an embodiment of the disclosure, the endoscope system includes a fourth optical fiber, a second optical fiber coupler, a first image sensor, and a second image sensor. The fourth optical fiber is connected to the bidirectional coupler. The second optical fiber coupler is connected to the fourth optical fiber, and an image beam received by the optical fiber endoscope is transmitted to the second optical fiber coupler through the bidirectional coupler, and the second optical fiber coupler is configured to divide the image beam into two sub-image beams. The first image sensor is configured to capture an image of one of the two sub-image beams with a wavelength between 400 nm and 800 nm. The second image sensor is configured to capture an image of the other one of the two sub-image beams with a wavelength between 900 nm and 1700 nm.

In an embodiment of the disclosure, the microlens array group includes a first microlens array and a second microlens array.

In an embodiment of the disclosure, the first microlens array includes a plurality of first microlenses, the second microlens array includes a plurality of second microlenses, the optical fiber endoscope includes a plurality of light source optical fibers and at least one image receiving optical fiber, the light source optical fibers correspond to the first microlenses, and the at least one image receiving optical fiber corresponds to the second microlenses.

In an embodiment of the disclosure, a radius of curvature of each of the first microlenses is smaller than a radius of curvature of each of the second microlenses.

In an embodiment of the disclosure, the at least one image receiving optical fiber is a plurality of image receiving optical fibers, and each of the image receiving optical fibers corresponds to one of the second microlenses.

In an embodiment of the disclosure, the light source optical fibers are arranged in a ring shape, and the image receiving optical fibers are surrounded by the light source optical fibers.

In an embodiment of the disclosure, the light source optical fibers are evenly distributed between the image receiving optical fibers.

In an embodiment of the disclosure, the at least one image receiving optical fiber is a single image receiving optical fiber, and the light source optical fibers are arranged in a ring shape, and the image receiving optical fiber is surrounded by the light source optical fibers.

In an embodiment of the disclosure, the endoscopic system further includes a third microlens array, a first optical filter, a fourth microlens array, and a second optical filter. The third microlens array is disposed between the second optical fiber coupler and the first image sensor. The first optical filter is disposed between the third microlens array and the first image sensor to allow the image with a wavelength between 400 nm and 800 nm to pass through. The fourth microlens array is disposed between the second optical fiber coupler and the second image sensor. The second optical filter is disposed between the fourth microlens array and the second image sensor to allow the image with a wavelength between 900 nm and 1700 nm to pass through.

In an embodiment of the disclosure, the endoscopic system further includes a third light source and a fifth optical fiber. A wavelength of light emitted by the third light source is between 900 nm and 1700 nm, and is different from the wavelength of light emitted by the second light source. The fifth optical fiber is connected between the third light source and the first optical fiber coupler.

In an embodiment of the disclosure, one of the wavelength of the light emitted by the second light source and the wavelength of the light emitted by the third light source is 1200 nm, and the other is 1550 nm.

In an embodiment of the disclosure, the endoscope system further includes a third light source, a fifth optical fiber and a third image sensor. A polarization state of light emitted by the third light source is different from polarization states of the light emitted by the first light source and the light emitted by the second light source. The fifth optical fiber is connected between the third light source and the first optical fiber coupler. The third image sensor is configured to capture a polarization image of another one of the sub-image beams.

In summary, the endoscope system of the disclosure comprises a first light source and a second light source, which are connected to a first optical fiber coupler through a first optical fiber and a second optical fiber, the first optical fiber coupler is connected to a bidirectional coupler through a third optical fiber, a second end of the optical fiber endoscope is connected to the bidirectional coupler, and a first end of the optical fiber endoscope is provided with a microlens array group. The endoscope system of the disclosure improves the quality of the endoscopic image of the captured affected part by using multiple light sources with different wavelengths.

### BRIEF DESCRIPTION OF THE DRAWINGS

The accompanying drawings are included to provide a further understanding of the disclosure, and are incorporated in and constitute a part of this specification. The drawings illustrate embodiments of the disclosure and, together with the description, serve to explain the principles of the disclosure.

FIG. 1 is a schematic diagram of an endoscope system according to an embodiment of the disclosure.

FIG. 2 is a schematic diagram of a first end of an optical fiber endoscope of the endoscope system of FIG. 1.

FIG. 3 is a schematic diagram of a microlens array group of the endoscope system of FIG. 1.

FIG. 4 is a partial schematic cross-sectional view of the first end of the optical fiber endoscope and a microlens array group of the endoscope system of FIG. 1.

FIG. 5 and FIG. 6 are schematic diagrams of first ends of optical fiber endoscopes of various endoscope systems of other embodiments of the disclosure.

FIG. 7 is a schematic diagram of an endoscope system according to another embodiment of the disclosure.

### DESCRIPTION OF THE EMBODIMENTS

Reference will now be made in detail to the present preferred embodiments of the disclosure, examples of which are illustrated in the accompanying drawings. Wherever possible, the same reference numbers are used in the drawings and the description to refer to the same or like parts.

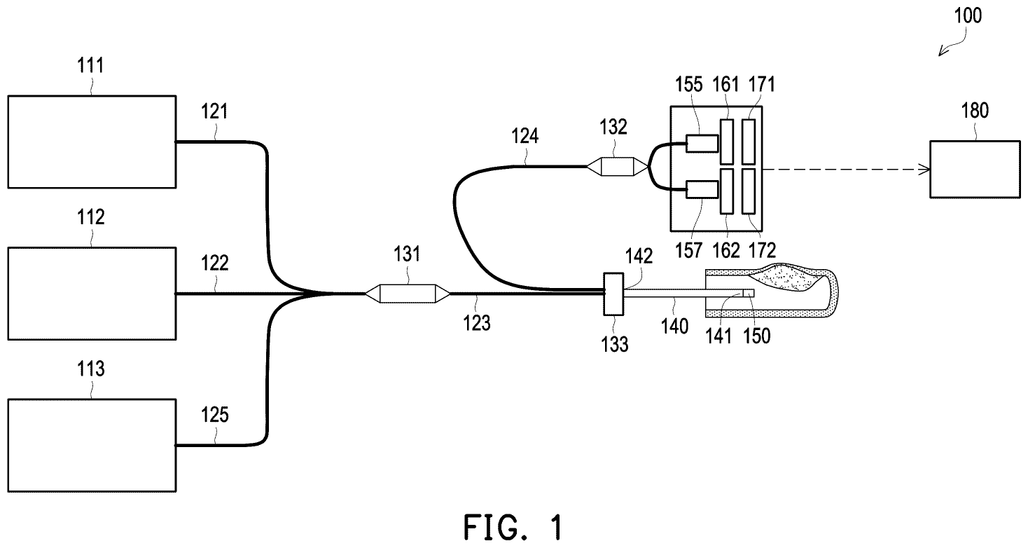

FIG. 1 is a schematic diagram of an endoscope system according to an embodiment of the disclosure. Referring to FIG. 1, an endoscope system 100 of the embodiment includes a first light source 111, a first optical fiber 121, a second light source 112, a second optical fiber 122, a first optical fiber coupler 131, a third optical fiber 123, a bidirectional coupler 133, and an optical fiber endoscope 140.

A wavelength of light emitted by the first light source 111 is between 400 nm and 800 nm. The first light source 111 is, for example, a halogen lamp, but the disclosure is not limited thereto. A wavelength of light emitted by the second light source 112 is between 900 nm and 1700 nm. The second light source 112 is, for example, a laser diode, but the disclosure is not limited thereto. In other embodiments, the second light source 112 may also be a short-wave infrared light emitting diode (SWIR LED).

In the embodiment, the first light source 111, for example, emits visible light, and emits light with a wavelength ranging from 400 nm to 800 nm, and the second light source 112, for example, emits short-wave infrared light, for example, light with a specific wavelength in a range between 900 nm and 1700 nm, such as 1200 nm.

Certainly, the types of the first light source 111 and the second light source 112 and the wavelengths of the emitted light are not limited thereto. The first light source 111 and the second light source 112 may also be a near-infrared light source or a polarized light source. Near-infrared light may be used with a fluorescent dye in the human, and the fluorescent dye is mainly Indocyanine green (ICG). The near-infrared light excites ICG to produce fluorescence, an excited wavelength of the near-infrared light is about 780 nm to 800 nm, and an emitted wavelength of the fluorescence is about 820 nm.

According to the characteristics of optics, light of different wavelengths may penetrate to different depths in different tissues of human body. The depth of light penetration is affected by a composition of layers of the tissues of each organ. For example, the depth of light penetration in a mucous tissue of a digestive tract is completely different from that in the skin on the outside of the body. For example, in terms of penetration depths of light of 400 nm to 1800 nm in the skin and the mucous tissue, the near-infrared light is deeper than the visible light and the short-wave infrared light.

For example, a wavelength of visible green light is about 500 nm, and a penetration depth thereof in the mucosal tissue is about 1 mm. However, if the near-infrared light of 800 nm is used to excite the ICG, an image thereof be taken to a depth of about 5 mm. Since a metabolic rate of the ICG in tumors and some tissues is slower than that in normal tissues, a larger amount of the ICG will remain in the tumors, which may be used to learn images of tumors deeper in mucosa, and to indicate blood vessels through a concentration difference of the ICG in blood vessels and tissues.

Therefore, through the characteristics that the near-infrared light is applied to ICG fluorescence, it may assist in learning a distribution of tumors in deeper regions. Moreover, polarization images of visible light or near-infrared light may also be used to provide more images of a boundary between mucosa and tumor.

Although the short-wave infrared light cannot reach the depth of the near-infrared light, and cannot effectively indicate specific cells or tumors like ICG, the short-wave infrared light may form a contrast display by using absorption characteristics of substances for special wavelengths through the technology of no need to add external developers.

In addition, the application of images of polarized light has its contribution to biological tissues, especially tumors and cardiovascular tissues. Human tissues are rich in collagen, and since collagen itself is a non-centrosymmetric material, it may cause the change in response to polarized light, the application of polarization images in endoscopy also has its value, which may help to provide boundary information for edges of tumors and plaques, so that doctors may better understand the judgment of a clearance boundary when performing treatments through the endoscope, and confirm whether a blood vessel wall is affected when removing plaques.

In other words, a designer may select a suitable type of light source according to the type of the diseased part to detect the diseased part better.

In addition, the first light source 111 and the second light source 112 may be turned on and off independently, so that the fiber endoscope 140 may be used to observe in real time images formed by reflecting lights of different wavelengths by a same part (the diseased part). Namely, the first light source 111 and the second light source 112 may be turned on at the same time or at different times, so that lights of different wavelengths may be used to detect the diseased part separately or together, and an actual situation of plaque (such as atherosclerosis) peeling treatment may be learned.

As shown in FIG. 1, the first optical fiber 121 is connected to the first light source 111. The second optical fiber 122 is connected to the second light source 112. The first optical fiber coupler 131 is connected to the first optical fiber 121 and the second optical fiber 122. The first optical fiber coupler 131 is, for example, a Y-type coupler or a tree coupler that couples light sources of multiple wavelengths together.

The third optical fiber 123 is connected to the first optical fiber coupler 131. The bidirectional coupler 133 is connected to the third optical fiber 123. The optical fiber endoscope 140 includes a first end 141 and a second end 142, where the first end 141 of the optical fiber endoscope 140 is provided with a microlens array group 150 so that a direction of light emitted from or received by the first end 141 of the optical fiber endoscope 140 may be more accurately oriented to a predetermined direction. The second end 142 of the optical fiber endoscope 140 is connected to the bidirectional coupler 133, so that light from the bidirectional coupler 133 may enter the optical fiber endoscope 140, and an image received from the optical fiber endoscope 140 may pass through the bidirectional coupler 133.

It should be noted that a diameter of a current cardiac catheter is about 1.7 to 2.3 mm, this is because the cardiac catheter usually enters human body through an artery at the wrist or groin, and an inner diameter of the artery at the wrist or groin is roughly the same as an outer diameter of the cardiac catheter. In addition, a main use of the cardiac catheter is mainly for coronary arteries, where blood vessels here are relatively thin. The optical fiber endoscope 140 of the disclosure also enters the human body through the wrist or groin to reduce bleeding, so that a designed outer diameter is mainly 1.5 to 2.5 mm, and the optical fiber endoscope 140 uses a flexible optical fiber as a light source guide for image acquisition. Since the endoscope enters the human body through the artery at the wrist or groin and goes up through the aorta to enter the heart, and the aorta has a large bend, the use of the flexible optical fiber may enable the optical fiber endoscope 140 to smoothly enter the coronary artery.

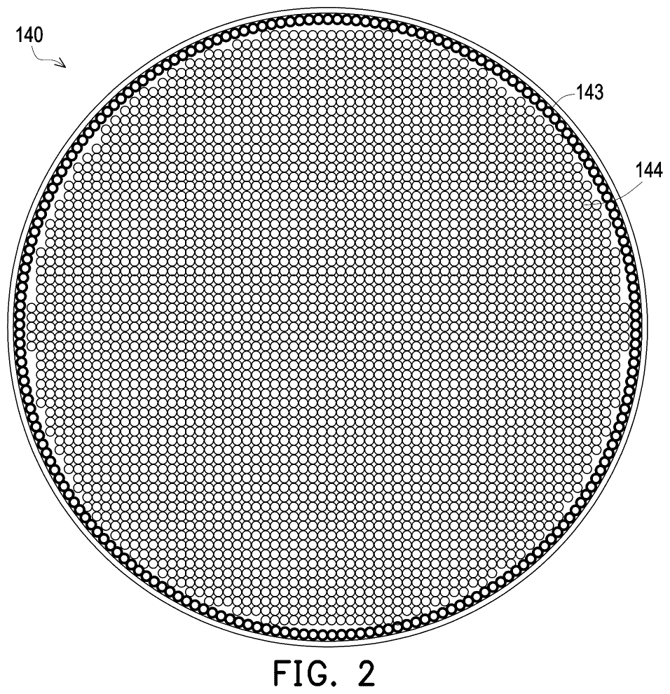

FIG. 2 is a schematic diagram of a first end of the optical fiber endoscope of the endoscope system of FIG. 1. It should be noted that in FIG. 2, light source optical fibers 143 are represented by thicker lines, and image receiving optical fibers 144 are represented by thinner lines.

Referring to FIG. 2, the optical fiber endoscope 140 includes light source optical fibers 143 and at least one image receiving optical fiber 144. In the embodiment, the at least one image receiving optical fiber 144 includes image receiving optical fibers 144. The light source optical fibers 143 are arranged in a ring shape, and the image receiving optical fibers 144 are surrounded by the light source optical fibers 143.

Due to the first optical fiber coupler 131, each of the light source optical fibers 143 of the optical fiber endoscope 140 may irradiate light of different wavelengths (at least including the light emitted by the first light source 111 and the second light source 112), and the image receiving optical fiber 144 may receive images formed through reflection of light of different wavelengths (at least including the light emitted by the first light source 111 and the second light source 112) after irradiating the diseased part. Since different wavelengths have different refractive indexes in a same material, total internal reflection angles of light of different wavelengths in the light source optical fibers 143 and the image receiving optical fibers 144 are different, so that there will be no interference problem.

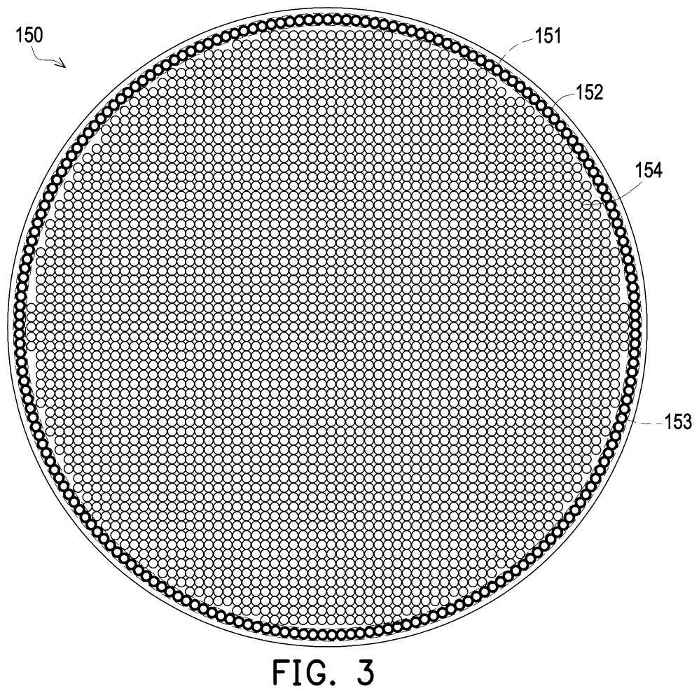

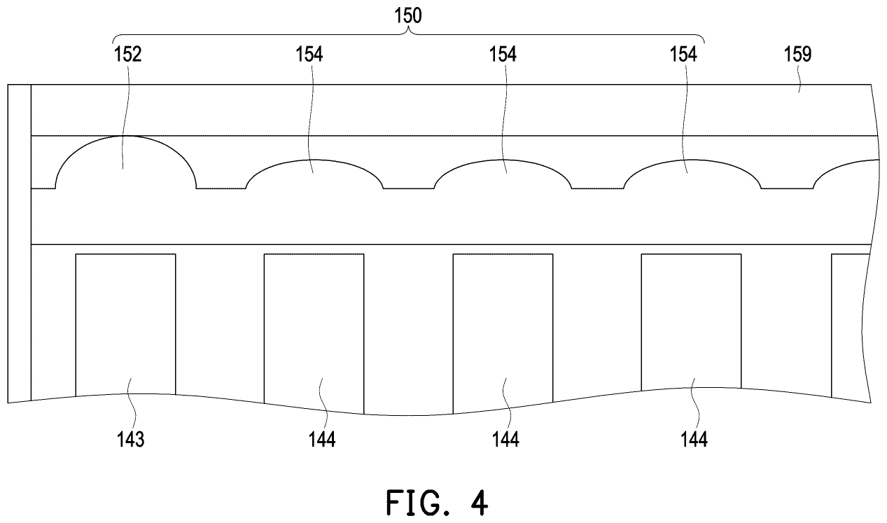

FIG. 3 is a schematic diagram of a microlens array group of the endoscope system of FIG. 1. FIG. 4 is a partial schematic cross-sectional view of the first end of the optical fiber endoscope and a microlens array group of the endoscope system of FIG. 1. It should be noted that in FIG. 3, first microlenses 152 are represented by thicker lines, and second microlenses 154 are represented by thinner lines.

As shown in FIG. 1, in the embodiment, the microlens array group 150 is disposed at a front side of the first end 141 of the optical fiber endoscope 140. Referring to FIG. 3 and FIG. 4, the microlens array group 150 includes a first microlens array 151 and a second microlens array 153.

The first microlens array 151 includes first microlenses 152, and the second microlens array 153 includes second microlenses 154. The first microlenses 152 are located at the outermost circle, and surround the second microlens array 153.

As shown in FIG. 2 to FIG. 4, the light source optical fibers 143 correspond to the first microlenses 152, and the image receiving optical fibers 144 correspond to the second microlenses 154. In the embodiment, each light source optical fiber 143 corresponds to one first microlens 152, and each image receiving optical fiber 144 corresponds to one second microlens 154. A cover plate 159 (FIG. 4) covers the microlens array group 150 to protect the microlens array group 150 and the fiber endoscope 140.

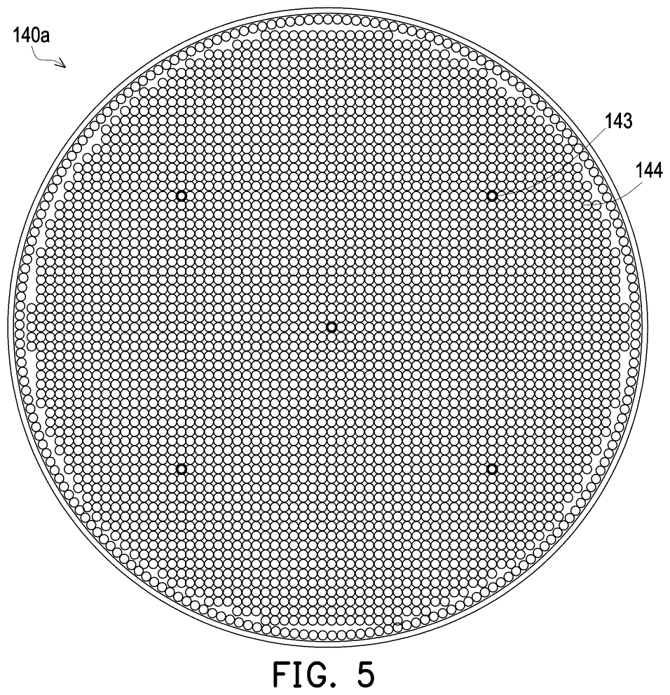

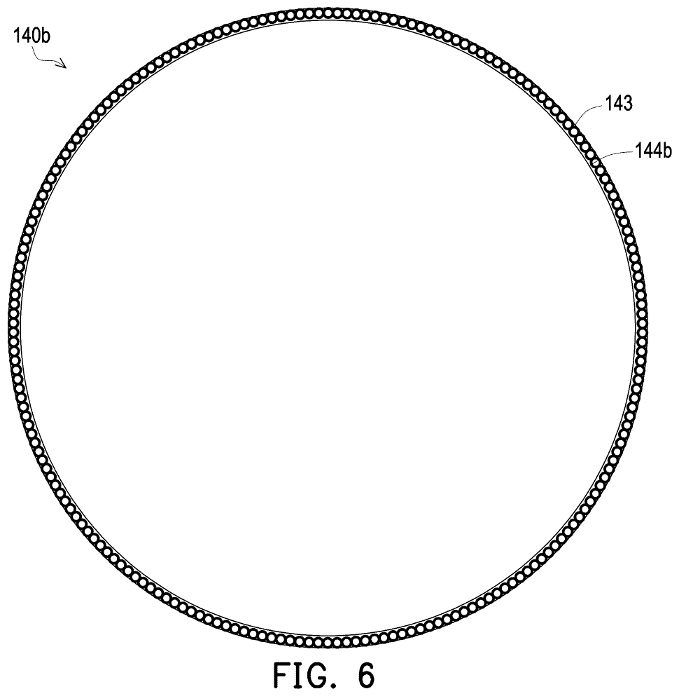

It should be noted that, as shown in FIG. 4, a radius of curvature of each of the first microlenses 152 is smaller than a radius of curvature of each of the second microlenses 154. Specifically, since the light source optical fiber 143 and the image receiving optical fiber 144 are used differently, focal lengths of the first microlens 152 and the second microlens 154 are different. Since the light source optical fiber 143 needs to irradiate a relatively large angle range, while the image receiving optical fiber 144 needs a relatively small angle range, the focal length of the light source optical fiber 143 needs to be relatively short, and therefore the radius of curvature of the first microlens 152 corresponding to the light source optical fiber 143 is relatively small. Configurations of the light source optical fiber and the image receiving optical fiber of other embodiments are introduced below. FIG. 5 and FIG. 6 are schematic diagrams of first ends of optical fiber endoscopes of various endoscope systems of other embodiments of the disclosure. It should be noted that in FIG. 5 and FIG. 6, the light source optical fibers 143 are represented by thicker lines, and the image receiving optical fibers 144 are represented by thinner lines. Referring to FIG. 5 first, a difference between FIG. 5 and FIG. 2 is that in FIG. 5, the light source optical fibers 143 of the fiber endoscope 140a are evenly distributed between the image receiving optical fibers 144.

It should be noted that, in the microlens array group corresponding to the optical fiber endoscope 140a in FIG. 5, each light source optical fiber 143 corresponds to a first microlens 152, and each image receiving optical fiber 144 corresponds to a second microlens 154. Namely, the first microlenses 152 are evenly distributed among the second microlenses 154. Furthermore, the radius of curvature of each of the first microlenses 152 is smaller than the radius of curvature of each of the second microlenses 154.

Referring to FIG. 6, in the embodiment, at least one image receiving optical fiber 144b of an optical fiber endoscope 140b is a single image receiving optical fiber 144b, the light source optical fibers 143 are arranged in a ring shape, and the image receiving optical fiber 144b is surrounded by the light source optical fibers 143. It should be noted that the microlens array group corresponding to the optical

fiber endoscope 140b of FIG. 6 may be the microlens array group 150 shown in FIG. 3. Namely, each light source optical fiber 143 corresponds to a first microlens 152, but the image receiving optical fiber 144 corresponds to the second microlenses 154. In addition, a radius of curvature of each of the first microlenses 152 is smaller than a radius of curvature of each of the second microlenses 154.

Certainly, the configurations of the light source optical fibers 143, the image receiving optical fibers 144, 144b, and the microlens array group 150 are not limited to the above.

Referring back to FIG. 1, the endoscope system 100 further includes a fourth optical fiber 124, a second optical fiber coupler 132, a first image sensor 171, and a second image sensor 172. The fourth optical fiber 124 is connected to the bidirectional coupler 133. The second optical fiber coupler 132 is connected to the fourth optical fiber 124. An image beam received by the fiber endoscope 140 is transmitted to the second optical fiber coupler 132 through the bidirectional coupler 133 and the fourth optical fiber 124, and the second optical fiber coupler 132 is used to split the image beam into two sub-image beams. The second optical fiber coupler 132 is, for example, a 50/50 split channel coupler, but the disclosure is not limited thereto.

The first image sensor 171 is used to capture an image of one of the two sub-image beams with a wavelength between 400 nm and 800 nm, and the second image sensor 172 is used to capture an image of the other one of the two sub-image beams with a wavelength between 900 nm and 1700 nm.

The second image sensor 172 is, for example, an image sensor based on InGaAs. A wavelength range of optical signals detected by the InGaAs image sensor is mainly 900 nm to 1700 nm, which belongs to short-wave infrared (SWIR) and is a wavelength range that cannot be detected by human eyes.

Some substances in the human body have special optical absorption characteristics in short-wave infrared (SWIR). For example, lipids have relative absorption peaks at about 1210 nm, 1430 nm and 1730 nm. However, since water (H2O) has a strong absorption peak at 1460 nm, 1430 nm and 1460 nm are close absorption bands, and 1730 nm is not within a detection band of the InGaAs image sensor, it is suitable to use a wavelength of 1210 nm as a light source for lipid detection and use the InGaAs image sensor for detection.

It should be noted that lipid accumulation is not unique to coronary arteries and cardiovascular system, but also exists in multiple organs of digestive tract and abdominal cavity. Therefore, the optical fiber endoscope 140 may be used not only for examinations of the coronary arteries and the cardiovascular system, but also for examinations of multiple organs of the digestive tract and abdominal cavity. In addition to the lipids, there are other substances in the body that have obvious absorption differences in short-wave infrared light. For example, the 1550 nm in short-wave infrared light may detect sugars, glucose, and may also observe vascular calcification and hardening. Therefore, the optical fiber endoscope 140 may be applied to a considerable number of detection contents and testing through the InGaAs image sensor.

In addition, as shown in FIG. 1, the endoscope system 100 further includes a third microlens array 155, a first optical filter 161, a fourth microlens array 157, and a second optical filter 162. The third microlens array 155 is disposed between the second optical fiber coupler 132 and the first image sensor 171. The fourth microlens array 157 is disposed between the second optical fiber coupler 132 and the second image sensor 172. The arrangement of the microlenses of the third microlens array 155 and the fourth microlens array 157 may correspond to pixel arrangement of the first image sensor 171 and the second image sensor 172, so that the two sub-image beams split by the second optical fiber coupler 132 may be focused onto the first image sensor 171 and the second image sensor 172.

The first optical filter 161 is disposed between the third microlens array 155 and the first image sensor 171 to allow images with wavelengths between 400 nm and 800 nm to pass through. The second optical filter 162 is disposed between the fourth microlens array 157 and the second image sensor 172 to allow images with wavelengths between 900 nm and 1700 nm to pass through. The images received by the first image sensor 171 and the second image sensor 172 are then transmitted to a calculator 180 for calculation.

It should be noted that the number of light sources is not limited to two. As shown in FIG. 1, in the embodiment, the endoscope system 100 may also selectively include a third light source 113 and a fifth optical fiber 125. A wavelength of the light emitted by the third light source 113 is between 900 nm and 1700 nm, and is different from the wavelength of the light emitted by the second light source 112. The fifth optical fiber 125 is connected between the third light source 113 and the first optical fiber coupler 131.

In an embodiment, one of the wavelength of the light emitted by the second light source 112 and the wavelength of the light emitted by the third light source 113 is 1200 nm, and the other is 1550 nm. 1200 nm is suitable for detecting lipids, and 1550 nm is suitable for detecting sugars. Certainly, the second light source 112 and the third light source 113 are not limited thereto.

The third light source 113 is connected to the fifth optical fiber 125, and the fifth optical fiber 125 is connected to the first optical fiber coupler 131. Therefore, in the embodiment, the first optical fiber coupler 131 may couple light sources of multiple wavelengths together, and transmit light to the optical fiber endoscope 140 through the bidirectional coupler 133, so as to irradiate the diseased part and receive an image of the diseased part. Thereafter, the image is received by the first image sensor 171 and the second image sensor 172 through the bidirectional coupler 133, the fourth optical fiber 124, and the second optical fiber coupler 132. In the embodiment, the images generated by the light emitted by the second light source 112 and the third light source 113 after irradiating the diseased part are received by the second image sensor 172. The images received by the first image sensor 171 and the second image sensor 172 are transmitted to the calculator 180 for processing.

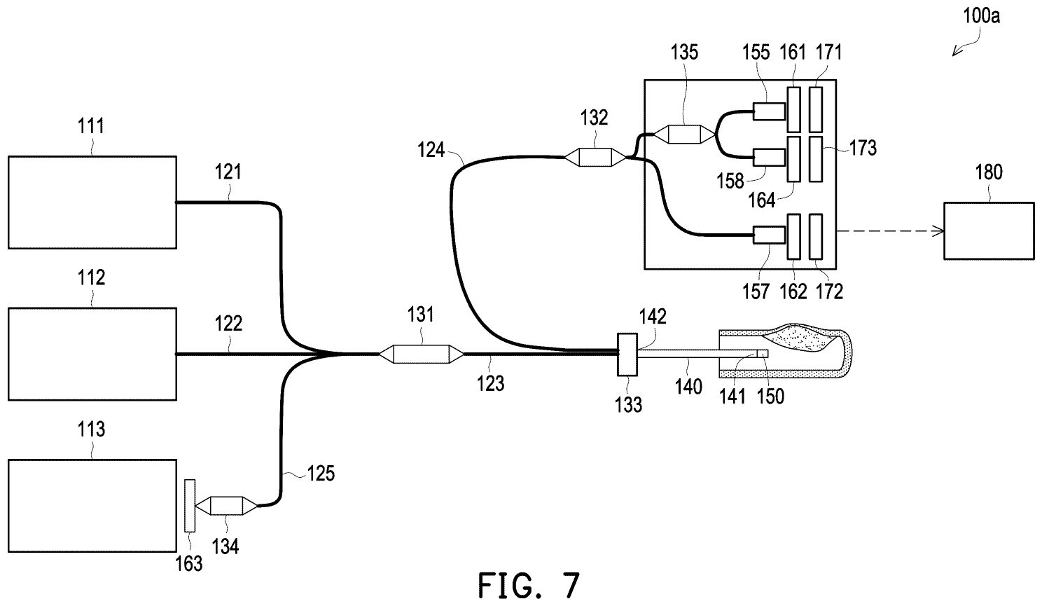

FIG. 7 is a schematic diagram of an endoscope system according to another embodiment of the disclosure. Referring to FIG. 7, a main difference between an endoscope system 100a of FIG. 7 and the endoscope system 100 of FIG. 1 is that, in the embodiment, the endoscope system 100a further includes polarization installation mechanisms 163, 164, a third optical fiber coupler 134, a fourth optical fiber coupler 135, a fifth microlens array 158 and a third image sensor 173, and the type of the third light source 113 is different.

For example, the endoscope system 100A of the embodiment is used to capture tumor images, and the first light source 111 may emit visible light, the second light source 112 may emit near-infrared light, and the third light source 113 may emit polarized light. In terms of arterial embolism, the first light source 111 may emit visible light, the second light source 112 may emit short-wave infrared, and the third light source 113 may emit polarized light. Certainly, the types of images obtained from the diseased part and the types of the light sources are not limited thereto.

In the embodiment, a polarization state of the light emitted by the third light source 113 is different from polarization states of the light emitted by the first light source 111 and the light emitted by the second light source 112. The third light source 113 may be a laser with polarization characteristics, or a light source without polarization (such as an LED or a halogen lamp) that is matched with the polarization installation mechanism 163 (such as a linear polarizer) to guide polarized light into the fifth optical fiber 125 through the third optical fiber coupler 134 and transmit it to the first optical fiber coupler 131.

The polarization installation mechanism 163 may be a polarizer with different phases on four adjacent pixels, which differ by 45 degrees from each other, so as to obtain polarization images of four angles. Alternatively, the polarization installation mechanism 163 may be a mechanism and assembly with a manual or automatic polarizer with a rotation function, which provides parameters of adjustable polarization angles according to each detection part, and the polarization installation mechanism 163 adjusts a direction to capture an expected polarization image. The polarization angle may be used to obtain an oscillation direction of an electric dipole of its structure, which obtains the maximum signal intensity in case of a parallel direction, and obtains the minimum signal intensity in case of a perpendicular direction.

On the other hand, an image captured through light reflected back to the optical fiber endoscope 140 after the polarized light irradiates the diseased part is transmitted to the second optical fiber coupler 132 through the bidirectional coupler 133, and the image beam is split into two sub-image beams. Then, one of the sub-image beams is further split into two by the fourth optical fiber coupler 135, one of which is received by the first image sensor 171 and the other is received by the third image sensor 173. The third image sensor 173 is, for example, a polarization image sensor.

The fifth microlens array 158 is disposed between the fourth optical fiber coupler 135 and the third image sensor 173, and the polarization installation mechanism 164 is disposed between the fifth microlens array 158 and the third image sensor 173.

Similarly, the polarization installation mechanism 164 may be a polarizer with different phases on four adjacent pixels, which differ by 45 degrees from each other, so as to capture polarization images of four angles. Alternatively, the polarization installation mechanism 164 may be a mechanism and assembly with a manual or automatic polarizer with a rotation function, which provides parameters of adjustable polarization angles according to each detection part, and the polarization installation mechanism 164 adjusts a direction to capture an expected polarization image. The polarization angle may be used to obtain an oscillation direction of an electric dipole of its structure, which obtains the maximum signal intensity in case of a parallel direction, and obtains the minimum signal intensity in case of a perpendicular direction. The polarization image at a specific angle may be received and integrated with visible light and other images for doctors to use. Certainly, the number, type and matching components of the light sources are not limited to the above.

In summary, the endoscope system of the disclosure comprises a first light source and a second light source, which are connected to a first optical fiber coupler through a first optical fiber and a second optical fiber, the first optical fiber coupler is connected to a bidirectional coupler through a third optical fiber, a second end of the optical fiber endoscope is connected to the bidirectional coupler, and a first end of the optical fiber endoscope is provided with a microlens array group. The endoscope system of the disclosure improves the quality of the endoscopic image of the captured affected part by using multiple light sources with different wavelengths.

Finally, it should be noted that the above embodiments are only used to illustrate the technical solution of the disclosure rather than limit it. Although the disclosure has been described in detail with reference to the foregoing embodiments, those of ordinary skill in the art should understand that: the technical solutions described in the foregoing embodiments may still be modified, or some or all of the technical features may be equivalently replaced; and these modifications or substitutions do not cause the essence of the corresponding technical solution to depart from the scope of the technical solution of each embodiment of the disclosure.

What is claimed is:

1. An endoscope system, comprising:
a first light source, wherein a wavelength of light emitted by the first light source is between 400 nm and 800 nm;
a first optical fiber, connected to the first light source;
a second light source, wherein a wavelength of light emitted by the second light source is between 900 nm and 1700 nm;
a second optical fiber, connected to the second light source;
a first optical fiber coupler, connected to the first optical fiber and the second optical fiber;
a third optical fiber, connected to the first optical fiber coupler;
a bidirectional coupler, connected to the third optical fiber; and
an optical fiber endoscope, having a first end and a second end, wherein the first end is provided with a microlens array group, and the second end is connected to the bidirectional coupler.

2. The endoscope system as claimed in claim 1, comprising:
a fourth optical fiber, connected to the bidirectional coupler;
a second optical fiber coupler, connected to the fourth optical fiber, wherein an image beam received by the optical fiber endoscope is transmitted to the second optical fiber coupler through the bidirectional coupler, and the second optical fiber coupler is configured to divide the image beam into a plurality of sub-image beams;
a first image sensor, configured to capture an image of one of the sub-image beams with a wavelength between 400 nm and 800 nm; and
a second image sensor, configured to capture an image of another one of the sub-image beams with a wavelength between 900 nm and 1700 nm.

3. The endoscope system as claimed in claim 1, wherein the microlens array group comprises a first microlens array and a second microlens array.

4. The endoscope system as claimed in claim 3, wherein the first microlens array comprises a plurality of first microlenses, the second microlens array comprises a plurality of second microlenses, the optical fiber endoscope comprises a plurality of light source optical fibers and at least one image receiving optical fiber, the light source optical fibers correspond to the first microlenses, and the at least one image receiving optical fiber corresponds to the second microlenses.

5. The endoscope system as claimed in claim 4, wherein a radius of curvature of each of the first microlenses is smaller than a radius of curvature of each of the second microlenses.

6. The endoscope system as claimed in claim 4, wherein the at least one image receiving optical fiber comprises a plurality of image receiving optical fibers, and each of the image receiving optical fibers corresponds to one of the second microlenses.

7. The endoscope system as claimed in claim 6, wherein the light source optical fibers are arranged in a ring shape, and the image receiving optical fibers are surrounded by the light source optical fibers.

8. The endoscope system as claimed in claim 6, wherein the light source optical fibers are evenly distributed between the image receiving optical fibers.

9. The endoscope system as claimed in claim 4, wherein the at least one image receiving optical fiber is a single image receiving optical fiber, the light source optical fibers are arranged in a ring shape, and the image receiving optical fiber is surrounded by the light source optical fibers.

10. The endoscope system as claimed in claim 2, further comprising:
a third microlens array, disposed between the second optical fiber coupler and the first image sensor;
a first optical filter, disposed between the third microlens array and the first image sensor to allow the image with the wavelength between 400 nm and 800 nm to pass through;
a fourth microlens array, disposed between the second optical fiber coupler and the second image sensor; and
a second optical filter, disposed between the fourth microlens array and the second image sensor to allow the image with the wavelength between 900 nm and 1700 nm to pass through.

11. The endoscope system as claimed in claim 1, further comprising:
a third light source, wherein a wavelength of light emitted by the third light source is between 900 nm and 1700 nm, and is different from the wavelength of the light emitted by the second light source; and
a fifth optical fiber, connected between the third light source and the first optical fiber coupler.

12. The endoscope system as claimed in claim 11, wherein one of the wavelength of the light emitted by the second light source and the wavelength of the light emitted by the third light source is 1200 nm, and the other is 1550 nm.

13. The endoscope system as claimed in claim 2, further comprising:
a third light source, a polarization state of light emitted by the third light source is different from polarization states of the light emitted by the first light source and the light emitted by the second light source;
a fifth optical fiber, connected between the third light source and the first optical fiber coupler; and
a third image sensor, configured to capture a polarization image of another one of the sub-image beams.
